(19) United States
(12) Patent Application Publication
(10) Pub. No.: US 2022/0390469 A1
(43) Pub. Date: Dec. 8, 2022

(54) DIAGNOSTIC METHODS FOR INFLAMMATORY DISORDERS

(71) Applicant: Meso Scale Technologies, LLC., Rockville, MD (US)

(72) Inventors: Jeffrey Debad, Gaithersburg, MD (US); Eli N. Glezer, Del Mar, CA (US); Sudeep Kumar, Hackettstown, NJ (US)

(73) Assignee: Meso Scale Technologies, LLC., Rockville, MD (US)

(21) Appl. No.: 17/890,782

(22) Filed: Aug. 18, 2022

Related U.S. Application Data
(62) Division of application No. 16/736,442, filed on Jan. 7, 2020, now abandoned.
Application No. 16/736,442 is a continuation of application No. 13/852,573, filed on Mar. 28, 2013, now abandoned.
(60) Provisional application No. 61/616,583, filed on Mar. 28, 2012.

(51) Int. Cl. G01N 33/68; G01N 33/574

(52) U.S. Cl. CPC G01N 33/6893; G01N 33/57423; G01N 2800/102; G01N 2800/122; G01N 2800/52; G01N 2800/324

(57) ABSTRACT

The present invention relates to methods of diagnosing an inflammatory disorder in a patient, as well as methods of monitoring the progression of an inflammatory disorder and/or methods of monitoring a treatment protocol of a therapeutic agent or regimen. The invention also relates to assay methods used in connection with the diagnostic methods described herein.

### CROSS REFERENCE TO RELATED APPLICATIONS

The present application is a divisional of U.S. patent application Ser. No. 16/736,442, which is a continuation of U.S. patent application Ser. No. 13/852,573 filed on Mar. 28, 2013, which claims benefit of U.S. Provisional Application No. 61/616,583 filed on Mar. 28, 2012, the entire contents of which are incorporated herein by reference.

### FIELD OF THE INVENTION

This application relates to assay methods, modules and kits for conducting diagnostic assays useful in the detection and treatment of inflammatory disorders.

### BACKGROUND OF THE INVENTION

A significant challenge in the field of inflammation is the lack of efficient diagnostic tools. Inflammatory conditions primarily rely on clinical evaluations and there are few or no useful in vitro biomarker assays available on the market to aid in diagnosis. It can be difficult for doctors to distinguish between certain disease states based on clinical evidence alone. For example, chronic obstructive pulmonary disorder (COPD) (which includes emphysema and chronic bronchitis) and asthma present with very similar symptoms such as shortness of breath, coughing, and wheezing, while shortness of breath is also a symptom found in coronary artery disease (CAD). Therefore, clinical evaluations are often unreliable for a definitive diagnosis of inflammatory conditions such as these.

### SUMMARY OF THE INVENTION

The invention provides a method for diagnosing chronic obstructive pulmonary disorder (COPD) in a patient suspected of having COPD, wherein the method includes (a) measuring a level of a first biomarker in a test sample obtained from a patient, wherein said first biomarker is selected from the group consisting of VEGF, ICAM-1, MCP-4, Thrombomodulin, P-selectin, bFGF, RANTES, and combinations thereof; and (b) diagnosing from said measuring step the presence or absence of COPD in said patient.

In another embodiment, the invention includes a method for monitoring the progression of COPD in a patient suspected of having COPD, said method comprising (a) measuring a level of a first biomarker in a test sample obtained from a patient, wherein said first biomarker is selected from the group consisting of VEGF, ICAM-1, MCP-4, Thrombomodulin, P-selectin, bFGF, RANTES, and combinations thereof; and (b) determining from said level(s) of said first biomarker the progression or efficacy of treatment of COPD in said patient.

The invention further provides a method for diagnosing rheumatoid arthritis (RA) in a patient suspected of having RA, said method comprising (a) measuring a level of a first biomarker in a test sample obtained from a patient, wherein said first biomarker is selected from the group consisting of TNF-RII, IL-6, TNF-R1, ICAM-1, TNF, CRP and VCAM-1; and (b) diagnosing from said measuring step the presence or absence of RA in said patient.

Also contemplated is a method for monitoring the progression of RA in a patient suspected of having RA, said method comprising (a) measuring a level of a first biomarker in a test sample obtained from a patient, wherein said first biomarker is selected from the group consisting of IL-6, TNF-RII, TNF-RI, TNF, ICAM-1, and combinations thereof; (b) determining from said level(s) of said first biomarker the progression or efficacy of treatment of RA in said patient.

Still further, the invention relates to a method for diagnosing coronary artery disease (CAD) in a patient suspected of having CAD, said method comprising (a) measuring a level of a first biomarker in a test sample obtained from a patient, wherein said first biomarker is selected from the group consisting of RANTES, Troponin-T, VCAM-1, cKit, PLGF, TNF, bFGF, CRP, IL-6 and ICAM-3; and (b) diagnosing from said measuring step the presence or absence of CAD in said patient.

Also provided is a method for monitoring the progression of CAD in a patient suspected of having CAD, said method comprising (a) measuring a level of a first biomarker in a test sample obtained from a patient, wherein said first biomarker is RANTES; and (b) determining from said level(s) of said first biomarker the progression or efficacy of treatment of CAD in said patient.

The invention also contemplates a method for diagnosing asthma in a patient suspected of having asthma, said method comprising (a) measuring a level of a first biomarker in a test sample obtained from a patient, wherein said first biomarker is selected from the group consisting of VEGF, bFGF, P-Selectin, PLGF, CRP, MCP-4, IL-12 (total), cKIT, IL-6R; and (b) diagnosing from said measuring step the presence or absence of asthma in said patient.

In addition, the invention includes a method for monitoring the progression of asthma in a patient suspected of having asthma, said method comprising (a) measuring a level of a first biomarker in a test sample obtained from a patient, wherein said first biomarker is VEGF; and (b) determining from said level(s) of said first biomarker the progression or efficacy of treatment of asthma in said patient.

Still further, the invention provides a method for evaluating the efficacy of a treatment regimen in a patient diagnosed with Chronic Obstructive Pulmonary Disorder (COPD), said method comprising

(a) obtaining a test sample from a patient undergoing said treatment regimen;

(b) measuring a level of a biomarker in said test sample, wherein said biomarker comprises VEGF, ICAM-1, MCP-4, Thrombomodulin, P-selectin, bFGF, RANTES, and combinations thereof;

(c) comparing said level to a normal control level of said biomarker; and

(d) evaluating from said comparing step (c) whether said patient is responsive to said treatment regimen.

Also provided is a method for evaluating the efficacy of a treatment regimen in a patient diagnosed with Rheumatoid Arthritis (RA), said method comprising

(a) obtaining a test sample from a patient undergoing said treatment regimen;

(b) measuring a level of a biomarker in said test sample, wherein said biomarker comprises TNF-RII, IL-6, TNF-R1, ICAM-1, TNF, and combinations thereof;

(c) comparing said level to a normal control level of said biomarker; and

(d) evaluating from said comparing step (c) whether said patient is responsive to said treatment regimen.

Moreover, the invention contemplates a method for evaluating the efficacy of a treatment regimen in a patient diagnosed with Coronary Artery Disease (CAD), said method comprising

(a) obtaining a test sample from a patient undergoing said treatment regimen;

(b) measuring a level of a biomarker in said test sample, wherein said biomarker comprises RANTES;

(c) comparing said level to a normal control level of said biomarker; and

(d) evaluating from said comparing step (c) whether said patient is responsive to said treatment regimen.

One embodiment of the invention is a method for evaluating the efficacy of a treatment regimen in a patient diagnosed with asthma, said method comprising

(a) obtaining a test sample from a patient undergoing said treatment regimen;

(b) measuring a level of a biomarker in said test sample, wherein said biomarker comprises VEGF;

(c) comparing said level to a normal control level of said biomarker; and

(d) evaluating from said comparing step (c) whether said patient is responsive to said treatment regimen.

The invention also includes a multiplexed assay kit configured to measure a level of a plurality of biomarkers in a patient sample, said plurality of biomarkers comprises VEGF, ICAM-1, MCP-4, Thrombomodulin, P-selectin, bFGF, RANTES, TNF-RII, IL-6, TNF-R1, TNF, CRP, VCAM-1, Troponin-T, cKit, PLGF, IL-12 (total), IL-6R, and ICAM-3 and combinations thereof.

Specific embodiments of the kit of the invention include:
a kit for the analysis of a COPD panel comprising (a) a multi-well assay plate comprising a plurality of wells, each well comprising at least four discrete binding domains to which capture antibodies to the following human analytes are bound: VEGF, ICAM-1, MCP-4, Thrombomodulin, P-selectin, bFGF, RANTES, and combinations thereof; (b) in one or more vials, containers, or compartments, a set of labeled detection antibodies specific for said human analytes; and (c) in one or more vials, containers, or compartments, a set of calibrator proteins;
a kit for the analysis of an RA panel comprising (a) a multi-well assay plate comprising a plurality of wells, each well comprising at least four discrete binding domains to which capture antibodies to the following human analytes are bound: TNF-RII, IL-6, TNF-R1, ICAM-1, TNF, CRP, VCAM-1, and combinations thereof; (b) in one or more vials, containers, or compartments, a set of labeled detection antibodies specific for said human analytes; and (c) in one or more vials, containers, or compartments, a set of calibrator proteins;
a kit for the analysis of a CAD panel comprising (a) a multi-well assay plate comprising a plurality of wells, each well comprising at least four discrete binding domains to which capture antibodies to the following human analytes are bound: RANTES, Troponin-T, VCAM-1, cKit, PLGF, TNF, bFGF, CRP, IL-6, ICAM-3, and combinations thereof; (b) in one or more vials, containers, or compartments, a set of labeled detection antibodies specific for said human analytes; and (c) in one or more vials, containers, or compartments, a set of calibrator proteins; and
a kit for the analysis of an asthma panel comprising (a) a multi-well assay plate comprising a plurality of wells, each well comprising at least four discrete binding domains to which capture antibodies to the following human analytes are bound: VEGF, bFGF, P-Selectin, PLGF, CRP, MCP-4, IL-12 (total), cKIT, IL-6R, and combinations thereof; (b) in one or more vials, containers, or compartments, a set of labeled detection antibodies specific for said human analytes; and (c) in one or more vials, containers, or compartments, a set of calibrator proteins.

One or more of the methods described herein may also include measuring a level of at least one additional biomarker in said sample and determining from said level of said first biomarker and said level of said at least one additional biomarker the presence or absence of said inflammatory condition in said patient.

### BRIEF DESCRIPTION OF THE FIGURES

FIG. 1 is a Receiver Operating Characteristic (ROC) curve analysis of VEGF levels in COPD patients.

FIG. 2 is an ROC curve analysis of MCP-4 levels in COPD patients.

FIG. 3 is an ROC curve analysis of ICAM-1 levels in COPD patients.

FIG. 4 is an ROC curve analysis of MCP-4 and VEGF in COPD patients.

FIG. 5 is an ROC curve analysis of TNF-RII levels in RA patients.

FIG. 6 is an ROC curve analysis of TNF-RI levels in RA patients.

FIGS. 7-8 are ROC curve analyses for all combinations of various marker levels (TNF-RII, IL-6, TNF-RI, ICAM-1, TNF, CRP and VCAM-1) in RA patients.

FIG. 9 shows an ROC curve analysis of various marker levels in serum samples from CAD patients.

FIG. 10 shows an ROC curve analysis for all combinations of various marker levels (RANTES, Troponin-T, VCAM-1, cKit, PLGF, TNF, bFGF, CRP, IL-6, ICAM-3, MPO, CKMB) in CAD patients.

FIG. 11 shows an ROC curve analysis of VEGF, TNF, MCP-1, MIP, and IL-8 levels in asthma patients.

FIG. 12 shows an ROC curve analysis for all combinations of various marker levels (VEGF, bFGF, P-Selectin, IL-6R, PLGF, CRP, MCP-4, IL-12total, MPO, cKit, IL-6, TNF-RI) in asthma patients.

FIGS. 13-16 are crossplots of various marker panels in COPD, CAD and/or asthma patients.

### DETAILED DESCRIPTION OF THE INVENTION

The present invention relates to the diagnosis of the chronic inflammatory diseases, including COPD, asthma, rheumatoid arthritis (RA) and CAD using biomarkers found in serum and/or plasma. The invention provides a method for diagnosing these inflammatory diseases using in vitro measurements of serum biomarkers. Still further, the invention provides a method of differentiating between inflammatory disorders that can present with similar clinical symptoms. As described in more detail below, the levels of certain individual biomarkers differ significantly for some inflammatory diseases and it has been found that the use of more than one biomarker measurement can improve the diagnosis for a given inflammatory disorder by improving the specificity and/or sensitivity of the diagnosis. Still further, different disorders can display significantly different patterns of biomarker elevation and/or suppression, and these differences can be used to provide a differential diagnosis for diseases.

In one embodiment, the invention provides a method of diagnosing COPD in a patient by measuring a level of one or more of the following markers in a sample: VEGF, ICAM-1, MCP-4, Thrombomodulin, P-Selectin, bFGF, and RANTES. In a preferred embodiment, the panel of markers may comprise one or more of VEGF, ICAM-1, and MCP-4. Still further, two separate panels may be analyzed, e.g., wherein one panel includes one or more of VEGF, ICAM-1, and MCP-4, and the other includes one or more of Thrombomodulin, P-Selectin, bGFG, and RANTES, and optionally, the results from each panel are compared. In a specific embodiment, levels of VEGF and MCP-4 are measured in the diagnostic method. Alternatively or additionally, levels of VEGF and ICAM-1 are measured as an aid in diagnosis.

The invention also provides a method of diagnosing asthma in a patient by measuring a level of VEGF, bFGF, P-Selectin, PLGF, CRP, MCP-4, IL-12 (total), cKIT, IL-6R, and combinations thereof. In a preferred embodiment, levels of VEGF, bFGF, P-Selectin, IL-6R, PLGF, and/or CRP are used in a panel to diagnose asthma. Most preferably, levels of VEGF, bFGF, and P-Selectin are analyzed as a diagnostic tool.

The invention further provides a method of diagnosing rheumatoid arthritis (RA) by measuring a level of one or more of TNF-RII, IL-6, TNF-R1, ICAM-1, TNF, CRP and VCAM-1. Preferably, levels of TNF-RII, IL-6, TNF-R1, ICAM-1, and combinations thereof are measured, and most preferably, TNF-RII and TNF-RI are measured.

Still further, the invention provides a method of diagnosing coronary artery disease (CAD) in a patient by measuring a level of RANTES, Troponin-T, VCAM-1, cKit, PLGF, TNF, bFGF, IL-6 and/or ICAM-3 in a patient. In a preferred embodiment, the levels of one or more of RANTES, VCAM-1, cKit, PLGF, TNF, bFGF, CRP are measured, and most preferably, the levels of RANTES, VCAM-1, cKit, bFGF, CRP and combinations thereof are measured.

Further, a comparison of various marker levels in a patient sample may be used to distinguish various inflammatory conditions. For example, one may determine the relative levels of MCP-4, VEGF, bFGF and P-Selectin in a sample to differentially diagnose COPD and asthma in a patient. In addition, one may distinguish between COPD and CAD on the basis of RANTES levels (which are elevated in COPD but depressed in CAD). In one embodiment of the present invention, the level of the first biomarker and/or the level of the additional biomarkers in the test sample are compared to the levels of these biomarkers in a corresponding normal control sample. The difference between the normal control sample biomarker levels and that of the test sample may be the basis for diagnosing an inflammatory condition in a patient. Alternatively, the method of the invention contemplates a comparison of the level of the first biomarker to a detection cut-off level, wherein the first biomarker level above or below the detection cut-off level is indicative of the inflammatory condition. In addition, the diagnostic methods of the invention also contemplate comparing the level of at least one additional biomarker to a detection cut-off level, wherein at least one additional biomarker level above or below the detection cut-off level is indicative of the inflammatory condition.

The assays of the present invention may be conducted by any suitable method. In one embodiment, the measuring step is conducted on a single sample, and it may also be conducted in a single assay chamber, including but not limited to a single well of an assay plate. The assay chamber may also be an assay chamber of a cartridge.

As used herein, the term “sample” is intended to mean any biological fluid, cell, tissue, organ or combinations or portions thereof, which includes or potentially includes a biomarker of a disease of interest. For example, a sample can be cells that are placed in or adapted to tissue culture. A sample further can be a subcellular fraction or extract, or a crude or substantially pure nucleic acid molecule or protein preparation. In one embodiment, the samples that may be analyzed in the assays of the present invention include but are not limited to blood or blood fractions such as, serum and plasma. The sample may also be a mucosal swab, e.g., a nasal, nasopharyngeal or throat swab. In one embodiment, the level is measured using an immunoassay.

As used herein, a “biomarker” is a substance that is associated with a particular disease. A change in the expression levels of a biomarker may correlate with the risk or progression of a disease or with the susceptibility of the disease to a given treatment. A biomarker may be useful in the diagnosis of disease risk or the presence of disease in an individual, or to tailor treatments for the disease in an individual (choices of drug treatment or administration regimes). In evaluating potential drug therapies, a biomarker may be used as a surrogate for a natural endpoint such as survival or irreversible morbidity. If a treatment alters a biomarker that has a direct connection to improved health, the biomarker serves as a “surrogate endpoint” for evaluating clinical benefit

A sample that is assayed in the diagnostic methods of the present invention may be obtained from any suitable patient, including but not limited to a patient suspected of having COPD, emphysema, chronic bronchitis, asthma, RA, CAD or a patient having a predisposition to one or more of these conditions. The patient may or may not exhibit symptoms associated with one or more of these conditions.

As used herein, the term “level” refers to the amount, concentration, accumulation or rate of a biomarker molecule. A level can be represented, for example, by the amount or synthesis rate of messenger RNA (mRNA) encoded by a gene, the amount or synthesis rate of polypeptide corresponding to a given amino acid sequence encoded by a gene, or the amount or synthesis rate of a biochemical form of a molecule accumulated in a cell, including, for example, the amount of particular post-synthetic modifications of a molecule such as a polypeptide, nucleic acid or small molecule. The term can be used to refer to an absolute amount of a molecule in a sample or to a relative amount of the molecule, including amount or concentration determined under steady-state or non-steady-state conditions. Level may also refer to an assay signal that correlates with the amount, concentration, accumulation or rate of change of a biomarker molecule. The expression level of a molecule can be determined relative to a control molecule in a sample.

According to one aspect of the invention, the levels or levels of biomarker(s) are measured in samples collected from individuals clinically diagnosed with or suspected of or at risk of developing an inflammatory or pre-inflammatory condition using conventional methods. For example, patients suspected of having COPD, asthma, and/or CAD may be diagnosed on the basis of the diagnostic methods of the present invention alone or in combination with conventional methods for diagnosing these disorders, including but not limited to lung function tests, chest X-ray, CT scan, MRI, EKG, and ECG. Similarly, patients suspected of having RA may be diagnosed on the basis of the diagnostic methods of the present invention alone or in combination with conventional RA diagnostic methods, including but not limited to the presence or absence of rheumatoid factor, citrulline antibody, and antinuclear antibody in an RA sample. The level or level(s) of biomarkers may also be used to screen for disease in a broad population of asymptomatic individuals. For example, specific biomarkers valuable in distinguishing between normal and diseased patients could be identified by visual inspection of the data, for example, data plotted on a one-dimensional or multidimensional graph, or using methods of statistical analysis, such as a statistically weighted difference between control individuals and diseased patients and/or Receiver Operating Characteristic (ROC) curve analysis.

For example and without limitation, diagnostically valuable biomarkers may be first identified using a statistically weighted difference between control individuals and diseased patients, calculated as

$$\frac{D - N}{\sqrt{{σ}_{D} * {σ}_{N}}}$$

wherein D is the median level of a biomarker in patients diagnosed as having, for example, inflammatory disease, N is the median of the control individuals, (σ_{D} is the standard deviation of D and σ_{N} is the standard deviation of N. The larger the magnitude, the greater the statistical difference between the diseased and normal populations.

According to one embodiment of the invention, biomarkers resulting in a statistically weighted difference between control individuals and diseased patients of greater than, e.g., 1, 1.5, 2, 2.5 or 3 could be identified as diagnostically valuable markers.

Another method of statistical analysis for identifying biomarkers is the use of z-scores, e.g., as described in Skates et al. (2007) Cancer EpidemioL Biomarkers Prevo 16(2):334-341.

Another method of statistical analysis that can be useful in the inventive methods of the invention for determining the efficacy of particular candidate analytes, such as particular biomarkers, for acting as diagnostic marker(s) is ROC curve analysis. An ROC curve is a graphical approach to looking at the effect of a cut-off criterion, e.g., a cut-off value for a diagnostic indicator such as an assay signal or the level of an analyte in a sample, on the ability of a diagnostic to correctly identify positive or negative samples or subjects. One axis of the ROC curve is the true positive rate (TPR, i.e., the probability that a true positive sample/subject will be correctly identified as positive, or alternatively, the false negative rate (FNR=1−TPR, the probability that a true positive sample/subject will be incorrectly identified as a negative). The other axis is the true negative rate, i.e., TNR, the probability that a true negative sample will be correctly identified as a negative, or alternatively, the false positive rate (FPR=1−TNR, the probability that a true negative sample will be incorrectly identified as positive). The ROC curve is generated using assay results for a population of samples/subjects by varying the diagnostic cut-off value used to identify samples/subjects as positive or negative and plotting calculated values of TPR or FNR and TNR or FPR for each cut-off value. The area under the curve (referred to herein as the ROC area) is one indication of the ability of the diagnostic to separate positive and negative samples/subjects.

Diagnostic indicators analyzed by ROC curve analysis may be a level of an analyte, e.g., a biomarker, or an assay signal. Alternatively, the diagnostic indicator may be a function of multiple measured values, for example, a function of the level/assay signal of a plurality of analytes, eg, a plurality of biomarkers, or a function that combines the level or assay signal of one or more analytes with a patient scoring value that is determined based on visual, radiological and/or histological evaluation of a patient. The multi-parameter analysis may provide more accurate diagnosis relative to analysis of a single marker.

Candidates for a multi-analyte panel could be selected by using criteria such as individual analyte ROC areas, median difference between groups normalized by geometric interquartile range (IQR) etc. The objective is to partition the analyte space to improve separation between groups (for example, normal and disease populations) or to minimize the misclassification rate.

One approach is to define a panel response as a weighted combination of individual analytes and then compute an objective function like ROC area, product of sensitivity and specificity, etc. See for e.g., WO 2004/058055, as well as US2006/0205012, the disclosures of which are incorporated herein by reference in their entireties.

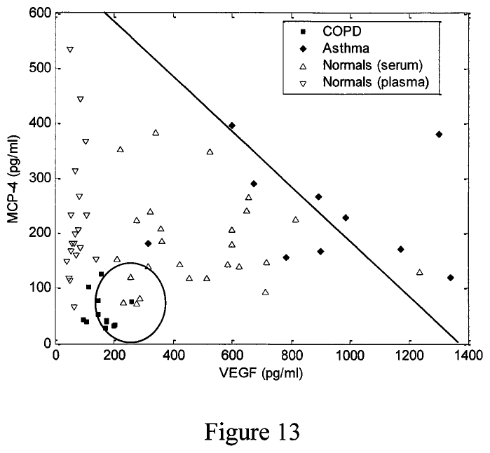

In one embodiment, the data is normalized (e.g., by the 99^{th} percentile of the normals), the centroid of normal and disease populations are identified and a separating plane that is perpendicular to the line joining the centroids is shifted to create the ROC curve (the centroid is evaluated by taking the mean along each assay axis, but a median or weighted average can also be used, especially for markers where the disease population has significant spread). This is shown in FIG. 13 for CAD. The normal data is shown by circles while the squares represent disease data. The filled symbols show the centroids of the two populations. In this case, using two assays (RANTES and CRP) improved the ROC area from 0.9 for RANTES to 0.985.

This approach may be applied to multi-dimensions and it provides a rapid method of searching for useful combinations of up to twenty five assays, e.g., up to fifteen assays, e.g., up to 10 assays, e.g., up to 5 assays (all combinations from 1 assay at a time to n assays at a time may be considered; this results in 33.6 million combinations for a group of twenty five assays). The partitioning object is a line in 2 dimensions, a plane in 3 dimensions and a hyperplane in higher dimensions. Candidate biomarkers for a panel can be selected by using criteria such as individual assay ROC areas, median difference normalized by geometric IQR etc.

This approach does not require the ‘sense’ of the marker to be known a priori, i.e., whether the biomarker level is increased or decreased in the diseased population. For example, in FIG. 13, RANTES levels were suppressed but CRP levels were increased in the disease state; this was identified by the algorithm. The relative weights of the assays are automatically determined by the angle of the separating line (and more generally by the normal to the separating hyperplane). For example, if the separating line is perpendicular to an assay axis, only that assay would be used to separate the two populations (this is equivalent to projecting the multi-dimensional data to that assay axis). In FIG. 13, the slope of the separating line is greater than 1; thus RANTES is weighted more than CRP (note that the weights are the differences between the two centroids; different approaches to calculating the centroid will provide different separating planes and therefore better coverage). Once a smaller set of assays is identified, the separating plane can be further optimized either by doing a dense search (e.g., by allowing rotation of the separating line at different increments along the line joining the centroids) or by using multi-dimensional methods for determining function extrema (downhill simplex methods, gradient descent methods and the like).

Once an operating point is chosen (e.g., by maximizing the product of sensitivity and specificity), different assay panels may be compared by evaluating a distance metric of the populations to the separating hyperplane. The algorithm is designed to find the best classification between two groups; it is therefore used to distinguish between different diseases or subgroups of the same disease. Finally, categorical data (age, gender, race etc) may also be coded into different levels and used as an optimizing variable in this process.

Biomarker levels may be measured using any of a number of techniques available to the person of ordinary skill in the art, e.g., direct physical measurements (e.g., mass spectrometry) or binding assays (e.g., immunoassays, agglutination assays and immunochromatographic assays). The method may also comprise measuring a signal that results from a chemical reaction, e.g., a change in optical absorbance, a change in fluorescence, the generation of chemiluminescence or electrochemiluminescence, a change in reflectivity, refractive index or light scattering, the accumulation or release of detectable labels from the surface, the oxidation or reduction or redox species, an electrical current or potential, changes in magnetic fields, etc. Suitable detection techniques may detect binding events by measuring the participation of labeled binding reagents through the measurement of the labels via their photoluminescence (e.g., via measurement of fluorescence, time-resolved fluorescence, evanescent wave fluorescence, up-converting phosphors, multi-photon fluorescence, etc.), chemiluminescence, electrochemiluminescence, light scattering, optical absorbance, radioactivity, magnetic fields, enzymatic activity (e.g., by measuring enzyme activity through enzymatic reactions that cause changes in optical absorbance or fluorescence or cause the emission of chemiluminescence). Alternatively, detection techniques may be used that do not require the use of labels, e.g., techniques based on measuring mass (e.g., surface acoustic wave measurements), refractive index (e.g., surface plasmon resonance measurements), or the inherent luminescence of an analyte.

Binding assays for measuring biomarker levels may use solid phase or homogenous formats. Suitable assay methods include sandwich or competitive binding assays. Examples of sandwich immunoassays are described in U.S. Pat. Nos. 4,168,146 and 4,366,241, both of which are incorporated herein by reference in their entireties. Examples of competitive immunoassays include those disclosed in U.S. Pat. Nos. 4,235,601, 4,442,204 and 5,208,535, each of which are incorporated herein by reference in their entireties.

Multiple biomarkers may be measured using a multiplexed assay format, e.g., multiplexing through the use of binding reagent arrays, multiplexing using spectral discrimination of labels, multiplexing of flow cytometric analysis of binding assays carried out on particles, e.g., using the Luminex® system. Suitable multiplexing methods include array based binding assays using patterned arrays of immobilized antibodies directed against the biomarkers of interest. Various approaches for conducting multiplexed assays have been described (See e.g., US 20040022677; US 20050052646; US 20030207290; US 20030113713; US 20050142033; and US 20040189311, each of which is incorporated herein by reference in their entireties. One approach to multiplexing binding assays involves the use of patterned arrays of binding reagents, e.g., U.S. Pat. Nos. 5,807,522 and 6,110,426; Delehanty J-B., Printing functional protein microarrays using piezoelectric capillaries, Methods Mol. Biol (2004) 278: 135-43; Lue R Y et al., Site-specific immobilization of biotinylated proteins for protein microarray analysis, Methods Mol. Biol. (2004) 278: 85-100; Lovett, Toxicogenomics: Toxicologists Brace for Genomics Revolution, Science (2000) 289: 536-537; Berns A, Cancer: Gene expression in diagnosis, nature (2000), 403, 491-92; Walt, Molecular Biology: Bead-based Fiber-Optic Arrays, Science (2000) 287: 451-52 for more details). Another approach involves the use of binding reagents coated on beads that can be individually identified and interrogated. See e.g., WO 9926067, which describes the use of magnetic particles that vary in size to assay multiple analytes; particles belonging to different distinct size ranges are used to assay different analytes. The particles are designed to be distinguished and individually interrogated by flow cytometry. Vignali has described a multiplex biding assay in which 64 different bead sets of microparticles are employed, each having a uniform and distinct proportion of two dyes (Vignali, D. A A, “Multiplexed Particle-Based Flow Cytometric Assays” J. ImmunoL Meth. (2000) 243: 243-55). A similar approach involving a set of 15 different beads of differing size and fluorescence has been disclosed as useful for simultaneous typing of multiple pneumococcal serotypes (Park, M. K et al., “A Latex Bead-Based Flow Cytometric Immunoassay Capable of Simultaneous Typing of Multiple Pneumococcal Serotypes (Multibead Assay)” Clin. Diagn. Lab ImmunoL (2000) 7: 486-9). Bishop, J E et al have described a multiplex sandwich assay for simultaneous quantification of six human cytokines (Bishop, L E. et al., “Simultaneous Quantification of Six Human Cytokines in a Single Sample Using Microparticle-based Flow Cytometric Technology,” Clin. Chem (1999) 45:1693-1694).

A diagnostic test may be conducted in a single assay chamber, such as a single well of an assay plate or an assay chamber that is an assay chamber of a cartridge. The assay modules, e.g., assay plates or cartridges or multi-well assay plates), methods and apparatuses for conducting assay measurements suitable for the present invention are described for example, in US 20040022677; US 20050052646; US 20050142033; US 20040189311, each of which is incorporated herein by reference in their entireties. Assay plates and plate readers are now commercially available (MULTISPOT® and MULTI-ARRAY® plates and SECTOR® instruments, Meso Scale Discovery®, a division of Meso Scale Diagnostics, LLC, Rockville, Md.).

The present invention relates to a kit for the analysis of a panel of target analytes. The kit is preferably configured to conduct a multiplexed assay of a plurality of analytes. The kit can include (a) a single panel arrayed on a multi-well plate which is configured to be used in an electrochemiluminescence assay, as well as (b) associated consumables, e.g., detection antibodies, calibrators, and optional diluents and/or buffers. Alternatively, the multi-well plates and associated consumables can be provided separately.

The panel is preferably configured in a multi-well assay plate including a plurality of wells, each well having an array with “spots” or discrete binding domains. Preferably, the array includes one, four, seven, ten, sixteen, or twenty-five binding domains, and most preferably, the array includes one, four, seven, or ten binding domains. A capture antibody to each analyte is immobilized on a binding domain in the well and that capture antibody is used to detect the presence of the target analyte in an immunoassay. Briefly, a sample suspected of containing that analyte is added to the well and if present, the analyte binds to the capture antibody at the designated binding domain. The presence of bound analyte on the binding domain is detected by adding labeled detection antibody. The detection antibody also binds to the analyte forming a “sandwich” complex (capture antibody-analyte-detection antibody) on the binding domain.

The multiplexed immunoassay kits described herein allow a user to simultaneously quantify multiple biomarkers. The panels are selected and optimized such that the individual assays function well together. The sample may require dilution prior to being assayed. Sample dilutions for specific sample matrices of interest are optimized for a given panel to minimize sample matrix effects and to maximize the likelihood that all the analytes in the panel will be within the dynamic range of the assay. In a preferred embodiment, all of the analytes in the panel are analyzed with the same sample dilution in at least one sample type. In another preferred embodiment, all of the analytes in a panel are measured using the same dilution for most sample types.

For a given panel, the detection antibody concentration and the number of labels per protein (L/P ratio) for the detection antibody are adjusted to bring the expected levels of all analytes into a quantifiable range at the same sample dilution. If one wants to increase the high end of the quantifiable range for a given analyte, then the L/P can be decreased and/or the detection antibody concentration is decreased. On the other hand, if one wants to increase the lower end of the quantifiable range, the L/P can be increased, the detection antibody concentration can be increased if it is not at the saturation level, and/or the background signal can be lowered.

Calibration standards for use with the assay panels are selected to provide the appropriate quantifiable range with the recommended sample dilution for the panel. The calibration standards have known concentrations of one of more of the analytes in the panel. Concentrations of the analytes in unknown samples are determined by comparison to these standards. In one embodiment, calibration standards comprise mixtures of the different analytes measured by an assay panel. Preferably, the analyte levels in a combined calibrator are selected such that the assay signals for each analyte are comparable, e.g., within a factor of two, a factor of five or a factor of 10. In another embodiment, calibration standards include mixtures of analytes from multiple different assay panels.

A calibration curve may be fit to the assay signals measured with calibration standards using, e.g., curve fits known in the art such as linear fits, 4-parameter logistic (4-PL) and 5-parameter (5-PL) fits. Using such fits, the concentration of analytes in an unknown sample may be determined by backfitting the measured assay signals to the calculated fits. Measurements with calibration standards may also be used to determine assay characteristics such as the limit of detection (LOD), limit of quantification (LOQ), dynamic range, and limit of linearity (LOL).

A kit can include the following assay components: a multi-well assay plate configured to conduct an immunoassay for one of the panels described herein, a set of detection antibodies for the analytes in the panel (wherein the set comprises individual detection antibodies and/or a composition comprising a blend of one or more individual detection antibodies), and a set of calibrators for the analytes in the panel (wherein the set comprises individual calibrator protein compositions and/or a composition comprising a blend of one or more individual calibrator proteins). The kit can also include one of more of the following additional components: a blocking buffer (used to block assay plates prior to addition of sample), an antibody diluent (used to dilute stock detection antibody concentrations to the working concentration), an assay diluent (used to dilute samples), a calibrator diluent (used to dilute or reconstitute calibration standards) and a read buffer (used to provide the appropriate environment for detection of assay labels, e.g., by an ECL measurement). The antibody and assay diluents are selected to reduce background, optimize specific signal, and reduce assay interference and matrix effect. The calibrator diluent is optimized to yield the longest shelf life and retention of calibrator activity. The blocking buffer should be optimized to reduce background. The read buffer is selected to yield the appropriate sensitivity, quantifiable range, and slowest off-rate. The reagent components of the kit can be provided as liquid reagents, lyophilized, or combinations thereof, diluted or undiluted, and the kit includes instructions for appropriate preparation of reagents prior to use. In a preferred embodiment, a set of detection antibodies are included in the kit comprising a plurality of individual detection antibody compositions in liquid form. Moreover, the set of calibrators provided in the kit preferably comprise a lyophilized blend of calibrator proteins. Still further, the kit includes a multi-well assay plate that has been pre-coated with capture antibodies and exposed to a stabilizing treatment to ensure the integrity and stability of the immobilized antibodies.

As part of a multiplexed panel development, assays are optimized to reduce calibrator and detection antibody non-specific binding. In sandwich immunoassays, specificity mainly comes from capture antibody binding. Some considerations for evaluating multiplexed panels include: (a) detection antibody non-specific binding to capture antibodies is reduced to lower background of assays in the panel, and this can be achieved by adjusting the concentrations and L/P of the detection antibodies; (b) non-specific binding of detection antibodies to other calibrators in the panel is also undesirable and should be minimized; (c) non-specific binding of other calibrators in the panel and other related analytes should be minimized; if there is calibrator non-specific binding, it can reduce the overall specificity of the assays in the panel and it can also yield unreliable results as there will be calibrator competition to bind the capture antibody.

Different assays in the panel may require different incubation times and sample handling requirements for optimal performance. Therefore, the goal is to select a protocol that's optimized for most assays in the panel. Optimization of the assay protocol includes, but is not limited to, adjusting one or more of the following protocol parameters: timing (incubation time of each step), preparation procedure (calibrators, samples, controls, etc.), and number of wash steps.

The reagents used in the kits, e.g., the detection and capture antibodies and calibrator proteins, are preferably subjected to analytical testing and meet or exceed the specifications for those tests. The analytical tests that can be used to characterize kit materials include but are not limited to, CIEF, DLS, reducing and/or non-reducing EXPERION, denaturing SDS-PAGE, non-denaturing SDS-PAGE, SEC-MALS, and combinations thereof. In a preferred embodiment, the materials are characterized by CIEF, DLS, and reducing and non-reducing EXPERION. One or more additional tests, including but not limited to denaturing SDS-PAGE, non-denaturing SDS-PAGE, SEC-MALS, and combinations thereof, can also be used to characterize the materials. In a preferred embodiment, the materials are also subjected to functional testing, i.e., a binding assay for the target analyte, as well as one or more characterization tests, such as those listed above. If the materials do not meet or exceed the specifications for the functional and/or characterization tests, they can be subjected to additional purification steps and re-tested. Each of these tests and the metrics applied to the analysis of raw materials subjected to these tests are described below:

Capillary Isoelectric Focusing (CIEF) is a technique commonly used to separate peptides and proteins, and it is useful in the detection of aggregates. During a CIEF separation, a capillary is filled with the sample in solution and when voltage is applied, the ions migrate to a region where they become neutral (pH=pI). The anodic end of the capillary sits in acidic solution (low pH), while the cathodic end sits in basic solution (high pH). Compounds of equal isoelectric points (pI) are “focused” into sharp segments and remain in their specific zone, which allows for their distinct detection based on molecular charge and isoelectric point. Each specific antibody solution will have a fingerprint CIEF that can change over time. When a protein solution deteriorates, the nature of the protein and the charge distribution can change. Therefore, CIEF is a particularly useful tool to assess the relative purity of a protein solution and it is a preferred method of characterizing the antibodies and calibrators in the plates and kits described herein. The metrics used in CIEF include pI of the main peak, the pI range of the solution, and the profile shape, and each of these measurements are compared to that of a reference standard.

Dynamic Light Scattering (DLS) is used to probe the diffusion of particulate materials either in solution or in suspension. By determining the rate of diffusion (the diffusion coefficient), information regarding the size of particles, the conformation of macromolecular chains, various interactions among the constituents in the solution or suspension, and even the kinetics of the scatterers can be obtained without the need for calibration. In a DLS experiment, the fluctuations (temporal variation, typically in a μs to ms time scale) of the scattered light from scatterers in a medium are recorded and analyzed in correlation delay time domain. Like CIEF, each protein solution will generate a fingerprint DLS for the particle size and it's ideally suited to detect aggregation. All IgGs, regardless of binding specificity, will exhibit the same DLS particle size. The metrics used to analyze a protein solution using DLS include percentage polydispersity, percentage intensity, percentage mass, and the radius of the protein peak. In a preferred embodiment, an antibody solution meets or exceeds one or more of the following DLS specifications: (a) radius of the antibody peak: 4-8 nm (antibody molecule size); (b) polydispersity of the antibody peak: <40% (measure of size heterogeneity of antibody molecules); (c) intensity of the antibody peak: >50% (if other peaks are present, then the antibody peak is the predominant peak); and (d) mass in the antibody peak: >50%.

Reducing and non-reducing gel electrophoresis are techniques well known in the art. The EXPERION™ (Bio-Rad Laboratories, Inc., www.bio-rad.com) automated electrophoresis station performs all of the steps of gel-based electrophoresis in one unit by automating and combining electrophoresis, staining, destaining, band detection, and imaging into a single step. It can be used to measure purity. Preferably, an antibody preparation is greater 50% pure by Experion, more preferably, greater than 75% pure, and most preferably greater than 80% pure. Metrics that are applied to protein analysis using non-reducing Experion include percentage total mass of protein, and for reducing Experion they include percentage total mass of the heavy and light chains in an antibody solution, and the heavy to light chain ratio.

Multi-Angle Light Scattering (MALS) detection can be used in the stand-alone (batch) mode to measure specific or non-specific protein interactions, as well as in conjunction with a separation system such as flow field flow fractionation (FFF) or size exclusion chromatography (SEC). The combined SEC-MALS method has many applications, such as the confirmation of the oligomeric state of a protein, quantification of protein aggregation, and determination of protein conjugate stoichiometry. Preferably, this method is used to detect molecular weight of the components of a sample.

As used herein, a lot of kits comprise a group of kits comprising kit components that meet a set of kit release specifications. A lot can include at least 10, at least 100, at least 500, at least 1,000, at least 5,000, or at least 10,000 kits and a subset of kits from that lot are subjected to analytical testing to ensure that the lot meets or exceeds the release specifications. In one embodiment, the release specifications include but are not limited to kit processing, reagent stability, and kit component storage condition specifications. Kit processing specifications include the maximum total sample incubation time and the maximum total time to complete an assay using the kit. Reagent stability specifications include the minimum stability of each reagent component of the kit at a specified storage temperature. Kit storage condition specifications include the range of storage temperatures for all components of the kit, the maximum storage temperature for frozen components of the kit, and the maximum storage temperature for non-frozen components of the kit. A subset of kits in a lot is reviewed in relation to these specifications and the size of the subset depends on the lot size. In a preferred embodiment, for a lot of up to 300 kits, a sampling of 4-7 kits are tested; for a lot of 300-950 kits, a sampling of 8-10 kits are tested; and for a lot of greater than 950 kits, a sampling of 10-12 kits are tested. Alternatively or additionally, a sampling of up to 1-5% preferably up to 1-3%, and most preferably up to 2% is tested.

In addition, each lot of multi-well assay plates is preferably subjected to uniformity and functional testing. A subset of plates in a lot is subjected to these testing methods and the size of the subset depends on the lot size. In a preferred embodiment, for a lot of up to 300 plates, a sampling of 4-7 plates are tested; for a lot of 300-950 plates, a sampling of 8-10 plates are tested; and for a lot of greater than 950 plates, a sampling of 10-12 plates are tested. Alternatively or additionally, a sampling of up to 1-5% preferably up to 1-3%, and most preferably up to 2% is tested. The uniformity and functional testing specifications are expressed in terms of % CV, Coefficient of Variability, which is a dimensionless number defined as the standard deviation of a set of measurements, in this case, the relative signal detected from binding domains across a plate, divided by the mean of the set.

One type of uniformity testing is protein A/G testing. Protein A/G binding is used to confirm that all binding domains within a plate are coupled to capture antibody. Protein A/G is a recombinant fusion protein that combines IgG binding domains of Protein A and protein G and it binds to all subclasses of human IgG, as well as IgA, IgE, IgM and, to a lesser extent, IgD. Protein A/G also binds to all subclasses of mouse IgG but not mouse IgA, IgM, or serum albumin, making it particularly well suited to detect mouse monoclonal IgG antibodies without interference from IgA, IgM, and serum albumin that might be present in the sample matrix. Protein A/G can be labeled with a detectable moiety, e.g., a fluorescent, chemiluminescent, or electrochemiluminescent label, preferably an ECL label, to facilitate detection. Therefore, if capture antibody is adhered to a binding domain of a well, it will bind to labeled protein A/G, and the relative amount of capture antibody bound to the surface across a plate can be measured.

In addition to the uniformity testing described above, a uniformity metric for a subset of plates within a lot can be calculated to assess within-plate trending. A uniformity metric is calculated using a matrix of normalized signals from protein A/G and/or other uniformity or functional tests. The raw signal data is smoothed by techniques known in the art, thereby subtracting noise from the raw data, and the uniformity metric is calculated by subtracting the minimum signal in the adjusted data set from the maximum signal.

In a preferred embodiment, a subset of plates in a lot is subjected to protein A/G and functional testing and that subset meet or exceed the following specifications:

TABLE 1
Plate Metrics

| | Preferred Specification for a subset of 96 well |
|---|---|
| Metric | multi-well plates |
| Average intraplate CV | ≤10% |
| Maximum intraplate CV | ≤13% |
| Average Uniformity | ≤25% |
| Maximum Uniformity | ≤37% |
| CV of intraplate averages | ≤18% |
| Signal, lower boundary | >1500 |
| Signal, upper boundary | <10⁽⁶⁾ |

As disclosed in U.S. Pat. No. 7,842,246 to Wohlstadter et al., the disclosure of which is incorporated herein by reference in its entirety, each plate consists of several elements, e.g., a plate top, a plate bottom, wells, working electrodes, counter electrodes, reference electrodes, dielectric materials, electrical connects, and assay reagents. The wells of the plate are defined by holes/openings in the plate top. The plate bottom can be affixed, manually or by automated means, to the plate top, and the plate bottom can serve as the bottom of the well. Plates may have any number of wells of any size or shape, arranged in any pattern or configuration, and they can be composed of a variety of different materials. Preferred embodiments of the invention use industry standard formats for the number, size, shape, and configuration of the plate and wells. Examples of standard formats include 96, 384, 1536, and 9600 well plates, with the wells configured in two-dimensional arrays. Other formats may include single well plates (preferably having a plurality of assay domains that form spot patterns within each well), 2 well plates, 6 well plates, 24 well plates, and 6144 well plates. Each well of the plate includes a spot pattern of varying density, ranging from one spot within a well to 2, 4, 7, 9, 10, 16, 25, etc., as described hereinabove.

Each plate is assembled according to a set of preferred specifications. In a preferred embodiment, a plate bottom meets or exceeds the following specifications:

TABLE 2
Plate bottom specifications

| | 96-well (round well) specifications in |
|---|---|
| Parameter | inches |
| Length range (C to C)* | 3.8904-3.9004 (A1-A12 and H1-H12) |
| Width range (C to C) | 2.4736-2.4836 (A1-A12 and H1-H12) |
| Well to well spacing | 0.3513-0.3573 |

*C to C well distance is the center of spot to center of spot distance between the outermost wells of a plate.

In a further preferred embodiment, the plate also meets or exceeds defined specifications for alignment of a spot pattern within a well of the plate. These specifications include three parameters: (a) Δx, the difference between the center of the spot pattern and the center of the well along the x axis of the plate (column-wise, long axis); (b) Δy, the difference between the center of the spot pattern and the center of the well along the y axis of the plate (row-wise, short axis); and (c) α, the counter-clockwise angle between the long axis of the plate bottom and the long axis of the plate top of a 96-well plate. In a preferred embodiment, the plate meets or exceeds the following specifications: Δx≤0.2 mm, Δy≤0.2 mm, and α≤0.1°.

The following non-limiting examples serve to illustrate rather than limit the present invention.

### EXAMPLES

A study was performed using serum/plasma from patients diagnosed with COPD, asthma, rheumatoid arthritis (RA) and coronary artery disease (CAD), and also with samples obtained from a normal, disease-free population. Forty three biomarkers, including cytokines, chemokines, inflammatory markers, vascular markers, cardiac markers, and growth factors were measured in each sample. As described in more detail below, the data were analyzed in a number of ways. First, biomarker levels were compared between diseased and normal populations and any significant differences in the levels of each biomarker between these populations were noted. Second, combinations of biomarker levels were analyzed to identify combinations of two or more biomarkers capable of improving the diagnosis. Still further, biomarkers or combinations of biomarkers were identified that were able to distinguish between disease states, i.e., the measurement of a certain combination of biomarkers could differentiate between one of the mentioned disease states and the other diseased states and the normal population.

For each assay described below, regardless of the patient population or the biomarker being analyzed, the following protocol was used to conduct an immunoassay of the concentration of the biomarker in the sample. A MULTI-SPOT® assay plate (available from MESO SCALE DISCOVERY®, Rockville Md.), e.g., a −24, −96, or −384 well MULTI-SPOT plate, was blocked for 1 hour using a suitable blocking solution, and subsequently washed using a washing buffer. Twenty five ul assay diluent were added to each well, followed by 25 ul calibrator or sample (undiluted or diluted) to each well of the MULTI-SPOT assay plate. The plate was incubated with shaking for about 1 to 2 hours and washed. Twenty five ul labeled antibody solution was added to each well and the plate was incubated with shaking for 1 to 2 hours, and subsequently washed. One hundred fifty ul read buffer was added to each well and the plate was read using an MSD plate reader (also available from MESO SCALE DISCOVERY).

### Example 1. Biomarkers for COPD

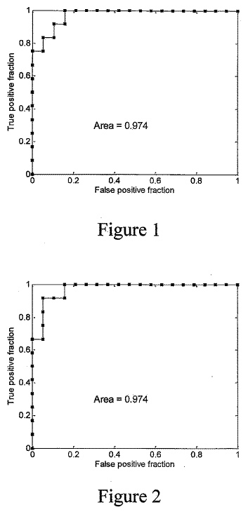

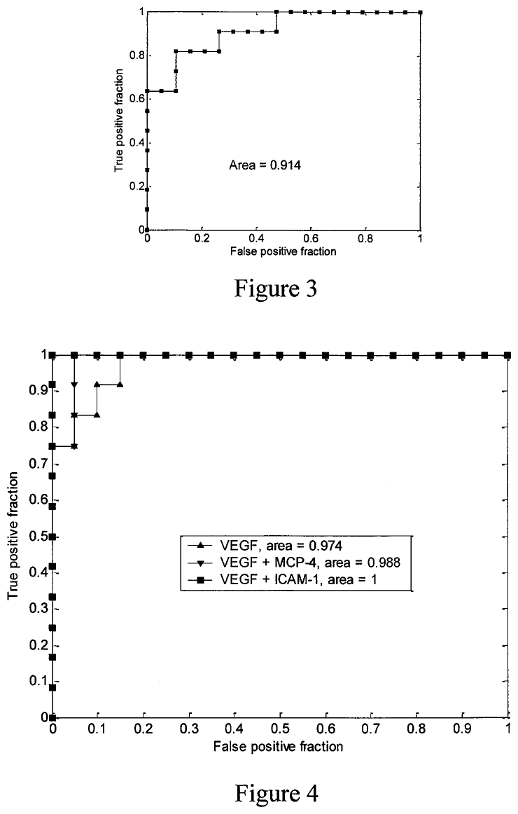

VEGF and ICAM-1 were strongly elevated while MCP-4 was depressed in plasma from COPD patients. Thrombomodulin, P-selectin, bFGF and RANTES were also elevated. The table below shows the results of assays with an ROC area 0.65. Assays with normal or disease medians within a factor of 2 of the detection limit are excluded. All data are in pg/ml except for CK-MB, Myoglobin, MPO (ng/ml) and CRP (ug/ml). Other things being equal, assays with a median difference greater than the geometric inter-quartile range (IQR) are preferred (data in last column of the table). The IQR is the difference between the 75th and the 25th percentile of the population. Unlike the standard deviation, it is a robust estimate of the spread of the data, since changes in the upper and lower 25% of the data do not affect it. The ROC figures for key assays are also shown in FIGS. 1-3 (FIG. 1 shows VEGF levels in COPD patients; FIG. 2 shows MCP-4 levels in COPD patients; and FIG. 3 shows ICAM-1 levels in COPD patients).

TABLE 3

| | | | | | | | Median |
|---|---|---|---|---|---|---|---|
| | | | | | | | delta/ |
| | ROC | Disease | Normal | Disease | Normal | | Geometric |
| Assay | area | samples | samples | median | median | DL | IQR |
| VEGF | 0.974 | 12 | 19 | 163.1 | 69.7 | 7.8 | 2.2 |
| MCP-4 | 0.974 | 12 | 19 | 42.3 | 180.7 | 2.8 | −2.1 |
| ICAM-1 | 0.914 | 11 | 19 | 514311.3 | 219260.8 | 402.5 | 1.8 |
| Thrombomodulin | 0.876 | 11 | 19 | 3319.1 | 2670.6 | 446.0 | 1.0 |
| P-Selectin | 0.871 | 11 | 19 | 130538.6 | 82688.4 | 5302.5 | 0.9 |
| bFGF | 0.833 | 12 | 19 | 13.7 | 9.7 | 1.6 | 1.6 |
| RANTES | 0.779 | 12 | 20 | 163013.6 | 76516.8 | 5050.0 | 0.7 |

As shown in FIG. 4, the use of MCP-4 in addition to VEGF improves the ROC area. A panel of VEGF and ICAM-1 gives notable separation between normal and disease samples with an ROC area of one (1).

### Example 2. Biomarkers for RA

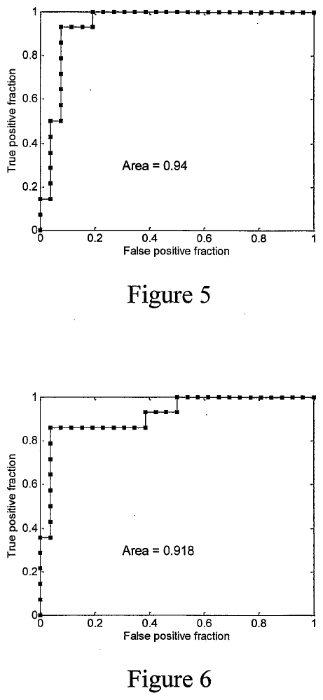

IL-6, TNF-RII, TNF-RI, TNF and ICAM-1 were found to be elevated in serum from RA patients as shown in Table 4 below. FIG. 5 shows the ROC curve for TNF-RII and FIG. 6 shows the ROC curve for TNF-RI.

TABLE 4

| | | | | | | | Median |
|---|---|---|---|---|---|---|---|
| | | | | | | | delta/ |
| | ROC | Disease | Normal | Disease | Normal | | Geometric |
| Assay | area | samples | samples | median | median | DL | IQR |
| TNF-RII | 0.94 | 14 | 26 | 11986.1 | 7766.6 | 550.0 | 1.8 |
| IL-6 | 0.929 | 13 | 26 | 7.4 | 1.6 | 0.2 | 1.0 |
| TNF-RI | 0.918 | 14 | 26 | 7171.5 | 5177.4 | 70.0 | 1.3 |
| ICAM-1 | 0.907 | 14 | 26 | 548013.9 | 325027.2 | 402.5 | 1.1 |
| TNF | 0.852 | 14 | 26 | 4.4 | 2.6 | 0.2 | 1.2 |
| CRP | 0.843 | 14 | 26 | 35.9 | 3.1 | 0.01 | 2.3 |
| VCAM-1 | 0.832 | 14 | 26 | 850156.5 | 561303.4 | 4875.0 | 1.2 |
| Troponin-T | 0.822 | 12 | 26 | 44.8 | 0.0 | 4.2 | 4.2 |
| bFGF | 0.794 | 14 | 26 | 11.1 | 16.5 | 1.6 | −1.1 |
| Thrombomodulin | 0.788 | 14 | 26 | 4309.4 | 3506.4 | 446.0 | 0.8 |
| ICAM-3 | 0.783 | 14 | 26 | 29576.7 | 19925.8 | 762.5 | 0.7 |
| s-Flt | 0.78 | 14 | 26 | 96.9 | 119.5 | 5.4 | −0.7 |
| E-Selectin | 0.766 | 14 | 26 | 34006.0 | 20551.1 | 746.0 | 0.8 |
| Eotaxin | 0.745 | 14 | 26 | 206.9 | 293.1 | 1.0 | −0.5 |
| CKMB | 0.731 | 14 | 26 | 0.5 | 0.9 | 0.1 | −0.7 |
| P-Selectin | 0.72 | 14 | 26 | 257351.6 | 371861.8 | 5302.5 | −0.7 |
| MDC | 0.72 | 14 | 26 | 950.1 | 682.1 | 14.0 | 0.6 |
| TARC | 0.706 | 14 | 26 | 252.9 | 198.2 | 3.2 | 0.3 |
| MCP-4 | 0.684 | 14 | 26 | 283.5 | 150.1 | 2.8 | 1.0 |
| IL-10 | 0.684 | 14 | 26 | 2.9 | 1.3 | 0.7 | 0.6 |

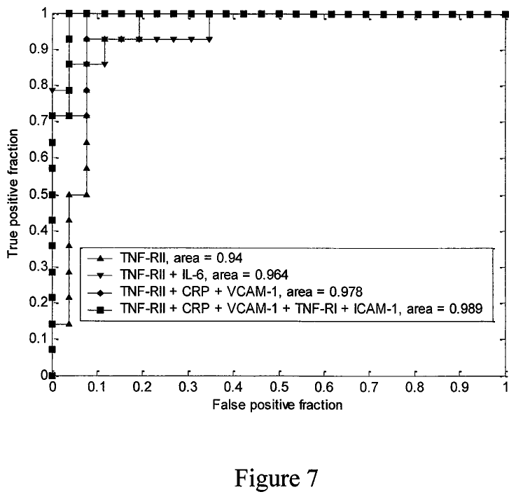

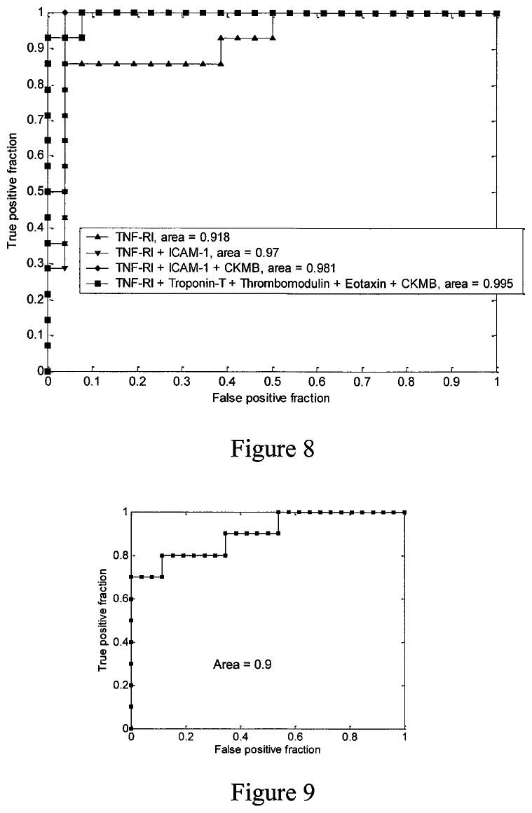

All combinations of seven assays were calculated (TNF-RII, IL-6, TNF-RI, ICAM-1, TNF, CRP and VCAM-1) and combinations with the highest ROC areas are shown in FIG. 7-8. If the top 15 assays from the 1D ROC table are used, several other combinations are found.

### Example 3. Biomarkers for CAD

Serum samples from CAD patients were tested and the most significant effect found was depression of RANTES levels as shown in Table 5. FIG. 9 shows the corresponding ROC curve.

TABLE 5

| | | | | | | | Median |
|---|---|---|---|---|---|---|---|
| | | | | | | | delta/ |
| | ROC | Disease | Normal | Disease | Normal | | Geometric |
| Assay | area | samples | samples | median | median | DL | IQR |
| RANTES | 0.9 | 10 | 26 | 91765.7 | 183208.4 | 5050.0 | −1.3 |
| Troponin-T | 0.868 | 9 | 26 | 6.8 | 0.0 | 4.2 | 2.9 |
| VCAM-1 | 0.796 | 10 | 26 | 687196.2 | 561303.4 | 4875.0 | 0.7 |
| cKit | 0.758 | 10 | 26 | 117286.1 | 147504.9 | 284.0 | −1.0 |
| PLGF | 0.75 | 10 | 26 | 21.7 | 20.0 | 0.7 | 0.3 |
| TNF | 0.75 | 10 | 26 | 3.9 | 2.6 | 0.2 | 0.8 |
| bFGF | 0.723 | 10 | 26 | 33.1 | 16.5 | 1.6 | 1.0 |
| CRP | 0.697 | 9 | 26 | 11.2 | 3.1 | 0.01 | 1.0 |
| IL-6 | 0.677 | 10 | 26 | 2.5 | 1.6 | 0.2 | 0.2 |
| ICAM-3 | 0.662 | 10 | 26 | 21851.4 | 19925.8 | 762.5 | 0.2 |

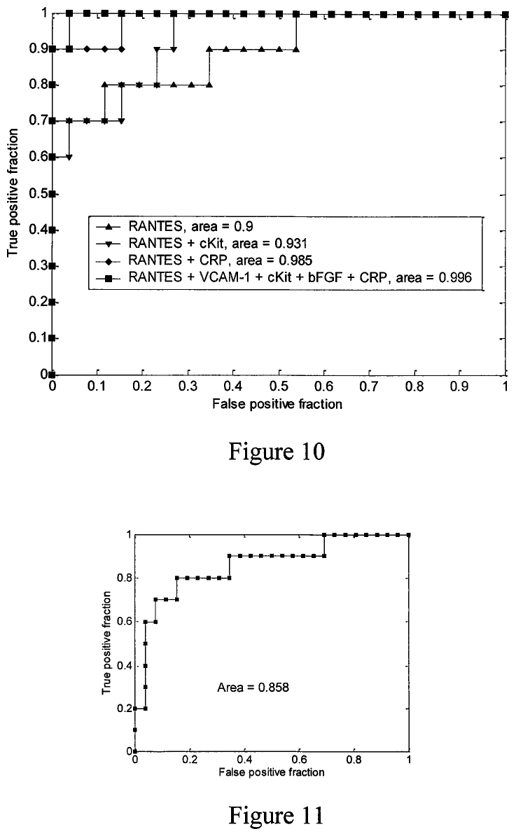

All combinations of 12 assays were calculated (RANTES, Troponin-T, VCAM-1, cKit, PLGF, TNF, bFGF, CRP, IL-6, ICAM-3, MPO, CKMB) and selected ROC curves are shown in FIG. 10.

### Example 4. Biomarkers for Asthma

Median VEGF levels showed a 2-fold increase in serum samples, as shown in Table 6, whereas cytokine markers identified in the literature as potentially relevant to the diagnosis of asthma, i.e., TNF, MCP-1, MIP, IL-8, were not elevated. FIG. 11 shows the corresponding ROC curve.

TABLE 6

| | | | | | | | Median |
|---|---|---|---|---|---|---|---|
| | | | | | | | delta/ |
| | ROC | Disease | Normal | Disease | Normal | | Geometric |
| Assay | area | samples | samples | median | median | DL | IQR |
| VEGF | 0.858 | 10 | 26 | 896.5 | 438.5 | 7.8 | 1.1 |
| bFGF | 0.819 | 10 | 26 | 34.6 | 16.5 | 1.6 | 0.9 |
| P-Selectin | 0.812 | 10 | 26 | 440752.0 | 371861.8 | 5302.5 | 0.4 |
| IL-6R | 0.731 | 10 | 26 | 23000.3 | 25631.4 | 3535.0 | −0.4 |
| PLGF | 0.731 | 10 | 26 | 17.6 | 20.0 | 0.7 | −0.7 |
| CRP | 0.722 | 9 | 26 | 10.2 | 3.1 | 0.0 | 1.0 |
| MCP-4 | 0.696 | 10 | 26 | 204.5 | 150.1 | 2.8 | 0.5 |
| IL-12(total) | 0.696 | 10 | 26 | 221.2 | 290.5 | 18.5 | −0.5 |
| MPO | 0.684 | 10 | 25 | 384.1 | 325.5 | 0.04 | 0.2 |
| cKit | 0.681 | 10 | 26 | 173608.2 | 147504.9 | 284.0 | 0.5 |
| IL-6 | 0.677 | 10 | 26 | 2.9 | 1.6 | 0.2 | 0.6 |
| TNF-RI | 0.65 | 10 | 26 | 5980.2 | 5177.4 | 70.0 | 0.4 |

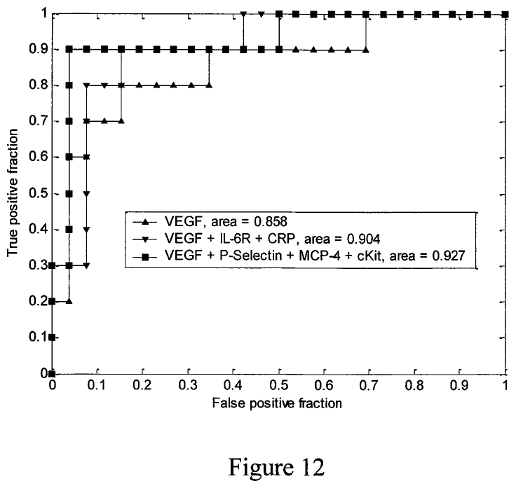

All combinations of 12 assays were calculated (VEGF, bFGF, P-Selectin, IL-6R, PLGF, CRP, MCP-4, IL-12total, MPO, cKit, IL-6, TNF-RI) and selected ROC curves are shown in FIG. 12.

### Example 5. Biomarker Panels to Distinguish Between Disease States

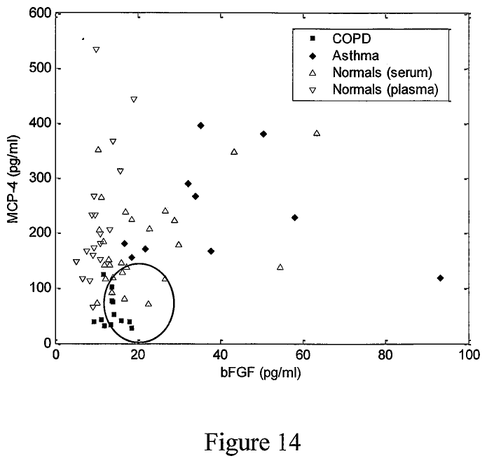

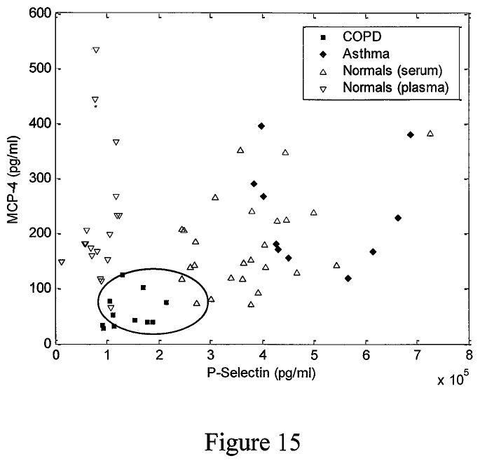

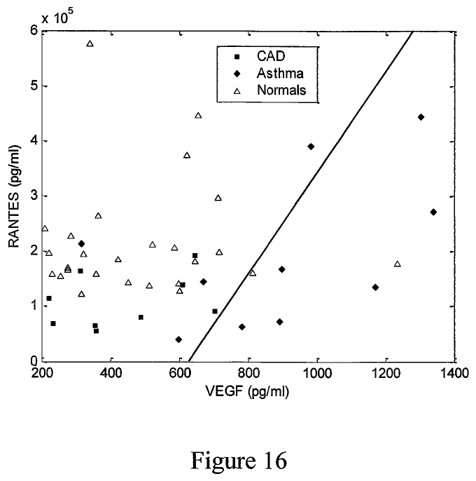

CAD and asthma often present with the same symptoms; hence, panels that can discriminate between the two are of particular utility. Crossplots of some panels are shown in FIGS. 13-16. For example, VEGF and MCP-4 can be used to discriminate between COPD and asthma as shown in FIG. 13, MCP-4 and bFGF can be used to discriminate between COPD and asthma as shown in FIG. 14, MCP-4 and P-Selectin can be used to discriminate between COPD and asthma as shown in FIG. 15, and RANTES and VEGF can be used to discriminate between CAD and asthma in FIG. 16.

Various publications and test methods are cited herein, the disclosures of which are incorporated herein by reference in their entireties, In cases where the present specification and a document incorporated by reference and/or referred to herein include conflicting disclosure, and/or inconsistent use of terminology, and/or the incorporated/referenced documents use or define terms differently than they are used or defined in the present specification, the present specification shall control.

### REFERENCES

1. Buist A S. Similarities and differences between asthma and chronic obstructive pulmonary disease: treatment and early outcomes. Eur Respir J Suppl. 2003 January; 39:30s-35s.
2. Tockman M S, Pearson J D, Fleg J L, Metter E J, Kao S Y, Rampal K G, Cruise L J, Fozard J L. Rapid decline in FEV1. A new risk factor for coronary heart disease mortality. Am J Respir Crit Care Med. 1995 February; 151(2 Pt 1):390-8.
3. Clin Exp Allergy. 2004 August; 34(8):1156-67. Cellular and molecular mechanisms in chronic obstructive pulmonary disease: an overview. Di Stefano A, Caramori G, Ricciardolo F L, Capelli A, Adcock I M, Donner C F.
4. Hua Xi Yi Ke Da Xue Xue Bao. 1999 September; 30(3):304-5, 309. Clinical usefulness of serum IFN-gamma level in patients with chronic obstructive pulmonary disease. [Article in Chinese] Chen B, Feng Y, Li S, Cai Y.
5. Eur Respir J. 2003 October; 22(4):602-8. Interleukin-13 and -4 expression in the central airways of smokers with chronic bronchitis. Miotto D, Ruggieri M P, Boschetto P, Cavallesco G, Papi A, Bononi I, Piola C, Murer B, Fabbri L M, Mapp C E.
6. Clin Appl Thromb Hemost. 2001 July; 7(3):205-8. Plasma markers of endothelial dysfunction in chronic obstructive pulmonary disease. Cella G, Sbarai A, Mazzaro G, Vanzo B, Romano S, Hoppensteadt T, Fareed J.
7. J Investig Med. 2000 January; 48(1):21-7. Soluble P-selectin as a marker of platelet hyperactivity in patients with chronic obstructive pulmonary disease. Ferroni P, Basili S, Martini F, Vieri M, Labbadia G, Cordova C, Alessandri C, Gazzaniga P P
8. J Korean Med Sci. 2004 June; 19(3):359-63. Acute and chronic changes of vascular endothelial growth factor (VEGF) in induced sputum of toluene diisocyanate (TDI)-induced asthma patients. Choi J H, Suh Y J, Lee S K, Suh C H, Nahm D H, Park H S.

1. A method for evaluating the efficacy of a treatment regimen in a patient diagnosed with Chronic Obstructive Pulmonary Disorder (COPD), said method comprising
(a) measuring a level of a biomarker in said test sample obtained from said patient undergoing said treatment regimen, wherein said biomarker comprises VEGF, ICAM-1, MCP-4, Thrombomodulin, P-selectin, bFGF, RANTES, and combinations thereof;
(b) comparing said level to a normal control level of said biomarker; and
(c) evaluating from said comparing step (b) whether said patient is responsive to said treatment regimen.

2. The method of claim 1 wherein said measuring step comprises conducting a multiplexed assay measurement of a plurality of said biomarkers in said test sample, wherein said multiplexed assay measurement is conducted using one reaction volume comprising said test sample.

3. The method of claim 1 wherein said method comprises measuring levels of two or more biomarkers.

4. The method of claim 1 further comprising one or more additional measuring steps including:
(x) measuring a baseline level(s) of said biomarker before said treatment regimen is initiated, and said evaluating step further comprises comparing said level and said baseline level; and
(y) measuring an interim level of said biomarker during said treatment regimen and said evaluating step further comprises comparing said level, said interim level and said baseline level.

5. The method of claim 1, wherein said evaluating step comprises comparing said level of said biomarker to a detection cut-off level, wherein said level below said detection cut-off level is indicative of lung cancer.

6. The method of claim 1 wherein said measuring step(s) are conducted in a single assay chamber.

7. The method of claim 1 wherein said level(s) are measured using an immunoassay.

8. A method for evaluating the efficacy of a treatment regimen in a patient diagnosed with Rheumatoid Arthritis (RA), said method comprising
(a) measuring a level of a biomarker in said test sample obtained from said patient undergoing said treatment regimen, wherein said biomarker comprises TNF-RII, IL-6, TNF-R1, ICAM-1, TNF, and combinations thereof;
(b) comparing said level to a normal control level of said biomarker; and
(c) evaluating from said comparing step (b) whether said patient is responsive to said treatment regimen.

9. The method of claim 8 wherein said measuring step comprises conducting a multiplexed assay measurement of a plurality of said biomarkers in said test sample, wherein said multiplexed assay measurement is conducted using one reaction volume comprising said test sample.

10. The method of claim 8 wherein said method comprises measuring levels of two or more biomarkers.

11. The method of claim 8 further comprising one or more additional measuring steps including:
(x) measuring a baseline level(s) of said biomarker before said treatment regimen is initiated, and said evaluating step further comprises comparing said level and said baseline level; and
(y) measuring an interim level of said biomarker during said treatment regimen and said evaluating step further comprises comparing said level, said interim level and said baseline level.

12. The method of claim 8, wherein said evaluating step comprises comparing said level of said biomarker to a detection cut-off level, wherein said level below said detection cut-off level is indicative of lung cancer.

13. The method of claim 8 wherein said measuring step(s) are conducted in a single assay chamber.

14. The method of claim 8 wherein said level(s) are measured using an immunoassay.

15. A method for evaluating the efficacy of a treatment regimen in a patient diagnosed with Coronary Artery Disease (CAD), said method comprising
(a) measuring a level of a biomarker in said test sample obtained from said patient undergoing said treatment regimen, wherein said biomarker comprises RANTES;
(b) comparing said level to a normal control level of said biomarker; and
(c) evaluating from said comparing step (b) whether said patient is responsive to said treatment regimen.

16. The method of claim 15 further comprising one or more additional measuring steps including:
(x) measuring a baseline level(s) of said biomarker before said treatment regimen is initiated, and said evaluating step further comprises comparing said level and said baseline level; and
(y) measuring an interim level of said biomarker during said treatment regimen and said evaluating step further comprises comparing said level, said interim level and said baseline level.

17. The method of claim 15, wherein said evaluating step comprises comparing said level of said biomarker to a detection cut-off level, wherein said level below said detection cut-off level is indicative of lung cancer.

18. The method of claim 15 wherein said measuring step(s) are conducted in a single assay chamber.

19. The method of claim 15 wherein said level(s) are measured using an immunoassay.

20. A method for evaluating the efficacy of a treatment regimen in a patient diagnosed with asthma, said method comprising
(a) measuring a level of a biomarker in said test sample obtained from said patient undergoing said treatment regimen, wherein said biomarker comprises VEGF;
(b) comparing said level to a normal control level of said biomarker; and
(c) evaluating from said comparing step (b) whether said patient is responsive to said treatment regimen.

21. The method of claim 20 further comprising one or more additional measuring steps including:
(x) measuring a baseline level(s) of said biomarker before said treatment regimen is initiated, and said evaluating step further comprises comparing said level and said baseline level; and
(y) measuring an interim level of said biomarker during said treatment regimen and said evaluating step further comprises comparing said level, said interim level and said baseline level.

22. The method of claim 20, wherein said evaluating step comprises comparing said level of said biomarker to a detection cut-off level, wherein said level below said detection cut-off level is indicative of lung cancer.

23. The method of claim 20 wherein said measuring step(s) are conducted in a single assay chamber.

24. The method of claim 20 wherein said level(s) are measured using an immunoassay.

25.-59. (canceled)
